(19) United States
(12) Patent Application Publication
(10) Pub. No.: US 2015/0182199 A1
(43) Pub. Date: Jul. 2, 2015

(54) ULTRASOUND DIAGNOSTIC APPARATUS AND METHOD FOR GENERATING DOPPLER SPECTRUM SIGNAL

(71) Applicant: GE Medical Systems Global Technology Company, LLC, Waukesha, WI (US)

(72) Inventor: Masafumi Ogasawara, Tokyo (JP)

(73) Assignee: GE Medical Systems Global Technology Company, LLC, Waukesha, WI (US)

(21) Appl. No.: 14/410,725

(22) PCT Filed: Jun. 10, 2013
(86) PCT No.: PCT/US13/44874

§ 371 Date: Dec. 23, 2014

(30) Foreign Application Priority Data
Jun. 29, 2012 (JP) 2012-146212

(51) Int. Cl. A61B 8/08; A61B 8/06

(52) U.S. Cl. CPC A61B 8/488; A61B 8/06; A61B 8/5207

(57) ABSTRACT

An ultrasound diagnostic apparatus includes: an ultrasound probe that performs ultrasound transmission/reception in Doppler mode and ultrasound transmission/reception in other mode than Doppler mode; and a Doppler processing unit that performs quadrature detection on an echo signal generated from the ultrasound transmission/reception for Doppler mode and then generates a Doppler spectrum signal. The Doppler processing unit includes a signal estimation unit that performs an extrapolation process to estimate missing part of the Doppler spectrum signal resulting from the ultrasound transmission/reception for other mode.

### TECHNICAL FIELD

The present invention relates to an ultrasound diagnostic apparatus that performs ultrasound transmission/reception for Doppler mode and ultrasound transmission/reception for other modes than Doppler mode such as B-mode and color Doppler mode, and a method for generating a Doppler spectrum signal.

### BACKGROUND ART

An ultrasound diagnostic apparatus displays images in various modes. For example, a Doppler mode image enables to observe a blood flow in a subject.

The ultrasound diagnostic apparatus may display a Doppler mode image along with a B-mode image or a color Doppler image. No ultrasound transmission/reception is performed in the Doppler mode while ultrasound transmission/reception is performed in the B-mode and the color Doppler. The Doppler image generation requires supplementing unavailability of signals for the ultrasound transmission/reception in the other modes than the Doppler mode.

There may be various techniques to estimate missing signals. For example, the technique described in patent document 1 simply uses a specified period before the beginning of a missing period as data for the missing period. Another technique decreases a sliding amount when a group of data after phase detection is read from the memory so that the data is used for frequency analysis according to FFT (Fast Fourier Transform). Still another technique drives an MA (moving average) filter using white noise.

[Patent Document 1]

JP-A No. 344971/1993 (FIG. 5 in paragraphs [0006] through [0008] on page 2)

### TECHNICAL PROBLEM

Any of the above-mentioned techniques can acquire a signal having sufficient quality when estimating missing part of a stationary signal. However, a signal having sufficient traceability is hardly estimated if the techniques estimate missing part of a non-stationary signal that varies with time. Therefore, a signal having sufficient quality cannot be acquired.

### SOLUTION TO PROBLEM

An aspect of the invention provides an ultrasound diagnostic apparatus that includes: an ultrasound probe that performs ultrasound transmission/reception in Doppler mode and ultrasound transmission/reception in other mode than Doppler mode; and a Doppler processing unit that performs quadrature detection on an echo signal generated from the ultrasound transmission/reception for Doppler mode and then generates a Doppler spectrum signal. The Doppler processing unit includes a signal estimation unit that performs an extrapolation process to estimate missing part of the Doppler spectrum signal resulting from the ultrasound transmission/reception for other mode.

### ADVANTAGEOUS EFFECTS OF INVENTION

According to the above-mentioned aspect of the invention, the Doppler processing unit performs an extrapolation process to estimate missing part of the Doppler spectrum signal resulting from the ultrasound transmission/reception for other mode than the Doppler mode. The missing part thereby becomes continuous with a part estimated by the extrapolation process. A high-quality signal is available even if the Doppler spectrum signal is not stationary.

Further objects and advantages of the present invention will be apparent from the following description of the preferred embodiments of the invention as illustrated in the accompanying drawings.

### BRIEF DESCRIPTION OF DRAWINGS

FIG. 1 is a block diagram illustrating a schematic configuration of an ultrasound diagnostic apparatus according to an embodiment of the invention;

FIG. 2 is a block diagram illustrating a schematic configuration of an echo data processing unit in the ultrasound diagnostic apparatus illustrated in FIG. 1;

FIG. 3 is a block diagram illustrating another schematic configuration of an echo data processing unit in the ultrasound diagnostic apparatus illustrated in FIG. 1;

FIG. 4 is a block diagram illustrating a configuration of a Doppler processing unit in the ultrasound diagnostic apparatus illustrated in FIG. 2 or 3;

FIG. 5 illustrates an ultrasound image displayed on a display unit;

FIG. 6 illustrates another ultrasound image displayed on a display unit;

FIG. 7 illustrates reading a group of data from the memory in the Doppler processing unit;

FIG. 8 illustrates an average frequency in the frequency spectrum for Doppler spectrum data;

FIG. 9 is a conceptual diagram illustrating Doppler spectrum data for an extrapolation process to supplement a missing part;

FIG. 10 is a conceptual diagram illustrating Doppler spectrum data with missing part supplemented by the extrapolation process;

FIG. 11 is a conceptual diagram illustrating Doppler spectrum data after termination of a missing period;

FIG. 12 illustrates a frequency having peak power in the frequency spectrum for Doppler spectrum data; and

FIG. 13 illustrates a maximum frequency in the frequency spectrum for Doppler spectrum data.

### DESCRIPTION OF EMBODIMENTS

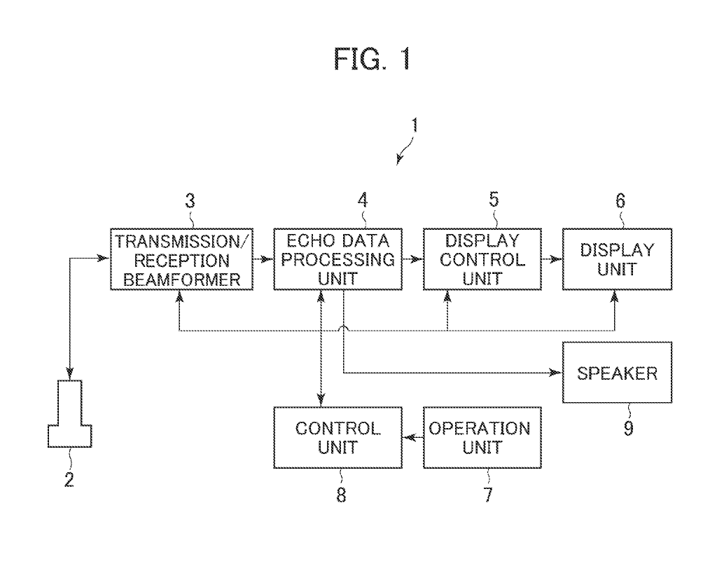

The embodiment of the invention will be described with reference to FIGS. 1 through 11. An ultrasound diagnostic apparatus 1 illustrated in FIG. 1 includes an ultrasound probe 2, a transmission/reception beamformer 3, an echo data processing unit 4, a display control unit 5, a display unit 6, an operation unit 7, a control unit 8, and a speaker 9.

The ultrasound probe 2 includes more than one ultrasound transducer (not shown) arranged in an array. The ultrasound transducer transmits ultrasound wave to a subject and receives an echo signal.

The transmission/reception beamformer 3 supplies an electric signal to the ultrasound probe 2 based on a control signal from the control unit 8 in order to transmit ultrasound wave from the ultrasound probe 2 using a specified parameter. The transmission/reception beamformer 3 performs signal processes such as amplification, A/D conversion, and phase rectifying addition on an echo signal received at the ultrasound probe 2 using a specified parameter. The transmission/reception beamformer 3 outputs processed echo data to the echo data processing unit 4. The transmission/reception beamformer 3 configures transmission/reception parameters according to modes such as the B-mode, the Doppler mode, and the color Doppler mode.

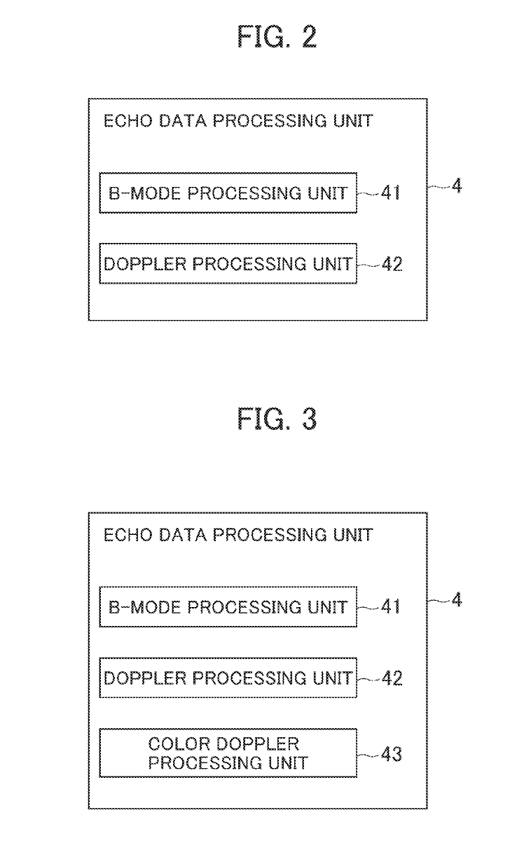

As illustrated in FIG. 2, the echo data processing unit 4 includes a B-mode processing unit 41 and a Doppler processing unit 42. As illustrated in FIG. 3, the echo data processing unit 4 may include the B-mode processing unit 41, the Doppler processing unit 42, and a color Doppler processing unit 43.

The echo data processing unit 4 generates B-mode data by performing B-mode processing such as logarithmic compression and envelope detection on echo data output from the transmission/reception beamformer 3. The color Doppler processing unit 43 generates color Doppler data by performing color Doppler processing such as quadrature detection, MTI (Moving Target Indication) filter processing, and autocorrelation processing.

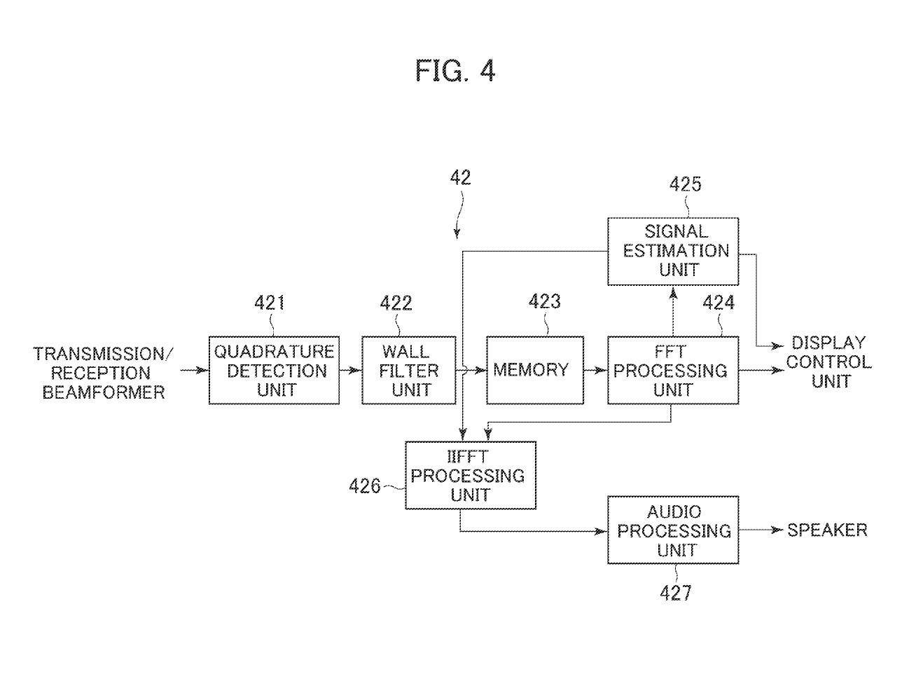

The Doppler processing unit 42 performs Doppler processing on the echo data to acquire a flow velocity spectrum such as a blood flow (Doppler processing function). As illustrated in FIG. 4, the Doppler processing unit 42 includes a quadrature detection unit 421, a wall filter unit 422, memory 423, an FFT processing unit 424, a signal estimation unit 425, an IFFT (Inverse Fast Fourier Transform) processing unit 426, and an audio processing unit 427. The detail will be described later.

The display control unit 5 uses a scan converter to convert data output from the echo data processing unit 4 into ultrasound image data by scanning. The display control unit 5 allows the display unit 6 to display an ultrasound image based on the ultrasound image data. The echo data processing unit 4 outputs B-mode data acquired from the B-mode processing unit 41, Doppler spectrum data acquired from the Doppler processing unit 42, and color Doppler data acquired from the color Doppler processing unit 43. The ultrasound image data includes B-mode image data, Doppler image data, and color Doppler image data. The display control unit 5 displays a B-mode image based on B-mode data, a Doppler image based on Doppler spectrum data, and a color Doppler image based on color Doppler data.

The display unit 6 includes an LCD (Liquid Crystal Display) or a CRT (Cathode Ray Tube). The operation unit 7 includes a keyboard and a pointing device (not shown) for an operator to enter an instruction or information.

The control unit 8 includes a CPU (Central Processing Unit). The control unit 8 reads a control program stored in a storage unit (not shown) and performs functions for the components of the ultrasound diagnostic apparatus 1.

The speaker 9 outputs Doppler sound based on a signal output from the echo data processing unit 4.

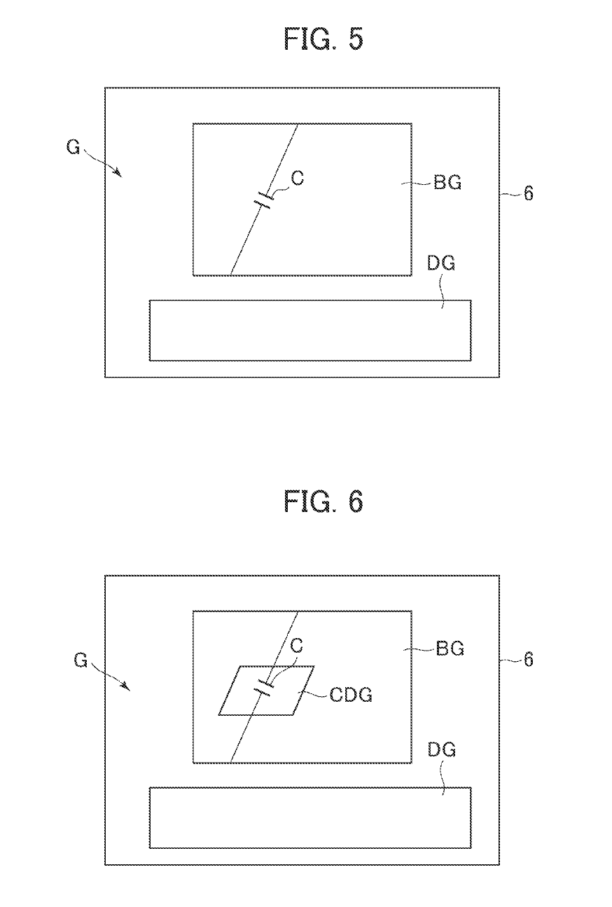

The following describes operations of the ultrasound diagnostic apparatus according to the example. The ultrasound probe 2 transmits and receives an ultrasound wave. Based on a resulting echo signal, the display unit 6 displays ultrasound image G. As illustrated in FIG. 5, the ultrasound image G may include B-mode image BG and Doppler image DG arranged vertically. As illustrated in FIG. 6, the ultrasound image G may include color Doppler image CDG overlaid on the B-mode image BG and the Doppler image DG arranged vertically.

In FIGS. 5 and 6, reference symbol C denotes a Doppler cursor.

If the B-mode image BG and the Doppler image DG are displayed as illustrated in FIG. 5, the control unit 8 outputs a control signal to the transmission/reception beamformer 3 to perform the ultrasound transmission/reception separately in the B-mode and the Doppler mode. If the B-mode image BG, the Doppler image DG, and the color Doppler image CDG are displayed as illustrated in FIG. 6, the control unit 8 outputs a control signal to the transmission/reception beamformer 3 to perform the ultrasound transmission/reception separately in the B-mode, the Doppler mode, and the color Doppler mode. For example, the control unit 8 outputs a control signal to the transmission/reception beamformer 3 so that the ultrasound transmission/reception in each mode becomes active according to each frame.

The Doppler mode includes PW (pulse wave) Doppler and CW (continuous wave) Doppler. The PW Doppler includes HPRF (High Pulse Repetition Frequency) Doppler.

The B-mode processing unit 41 generates B-mode data based on an echo signal acquired from the ultrasound transmission/reception in the B-mode. The Doppler processing unit 42 generates Doppler spectrum data based on an echo signal acquired from the ultrasound transmission/reception in the Doppler mode. The color Doppler processing unit 43 generates color Doppler data based on an echo signal acquired from the ultrasound transmission/reception in the color Doppler mode.

The following describes in detail signal processing of the Doppler processing unit 42. The transmission/reception beamformer 3 inputs data to the Doppler processing unit 42. As illustrated in FIG. 4, the data is first input to the quadrature detection unit 421. The quadrature detection unit 421 performs quadrature detection on the input data. The wall filter unit 422 filters the data to generate Doppler data. The Doppler data output from the wall filter unit 422 is stored in the memory 423.

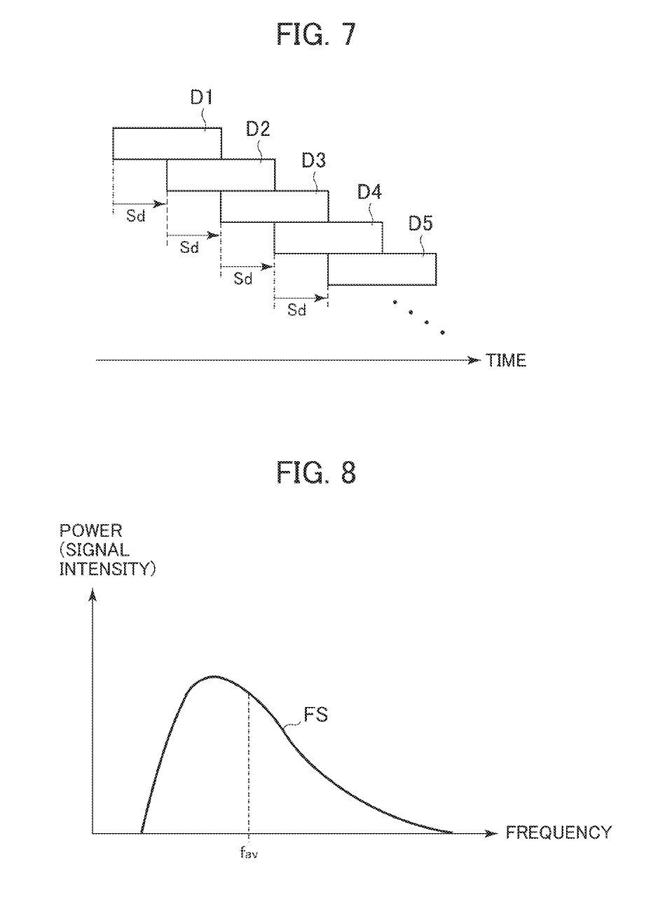

The memory 423 is equivalent to a sliding ring-buffer, for example. A group of data D1, D2, D3, D4, D5, and so on, for FFT processing is read from the memory 423 so as to maintain specified sliding amount Sd as illustrated in FIG. 7. The data is then input to the FFT processing unit 424.

The FFT processing unit 424 performs FFT processing on data supplied from the memory 423 to generate Doppler spectrum data. If missing part estimation is not performed on the Doppler spectrum data, the FFT processing unit 424 outputs the Doppler spectrum data to the display control unit 5 and the IFFT processing unit 426. If missing part estimation is performed on the Doppler spectrum data, the FFT processing unit 424 outputs the Doppler spectrum data to the signal estimation unit 425. Namely, the FFT processing unit 424 separates output of the Doppler spectrum data to the display control unit 5 and the IFFT processing unit 426 from output of the Doppler spectrum data to the signal estimation unit 425.

The signal estimation unit 425 estimates missing part of the Doppler spectrum data (signal estimation function). Missing part of the Doppler spectrum data occurs during a period in which the ultrasound transmission/reception in the B-mode or the color Doppler mode is performed and the ultrasound transmission/reception in the Doppler mode is not performed.

The signal estimation unit 425 uses an extrapolation process to estimate missing part of the Doppler spectrum data. As illustrated in FIG. 8, the signal estimation unit 425 according to the example performs the extrapolation process based on a temporal change of average frequency fav in frequency spectrum FS for the Doppler spectrum data.

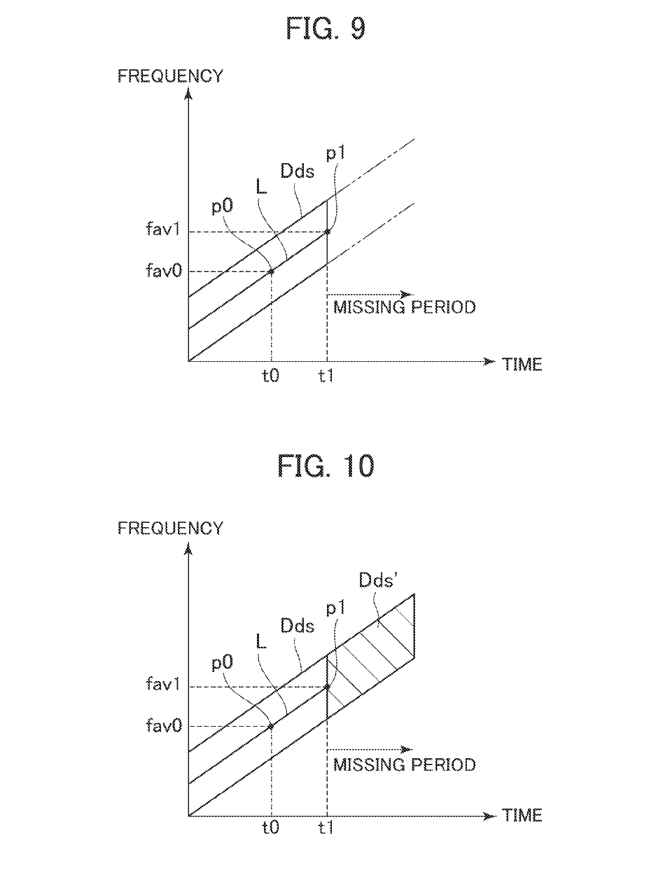

Specifically, as illustrated in FIG. 9, Doppler spectrum data Dds is acquired up to time t1. At time t1 and later, a missing period for Doppler spectrum data Dds begins. The signal estimation unit 425 performs an extrapolation process based on temporal change line L for average frequency fav in Doppler spectrum data Dds. For example, the signal estimation unit 425 performs an extrapolation process using linear function F as an extrapolation function. Linear function F is found from two points in a data string on change line L for average frequency fav. Two points in a data string on change line L include point p1 (average frequency fav1) at time t1 and point p0 (average frequency fav0) at t0 earlier than time t1. The width supplemented by the extrapolation process in the frequency axis (velocity axis) direction corresponds to the width of Doppler spectrum data Dds in the frequency axis (velocity axis) direction at time t1 immediately before the beginning of the missing period.

A temporal change degree (waveform) of Doppler spectrum data Dds depends on subject regions. Therefore, the signal estimation unit 425 may configure an interval of data (an interval between points p0 and p1) to find the extrapolation function according to subject regions so as to perform an extrapolation process that improves the signal quality according to temporal change degrees of Doppler spectrum data Dds.

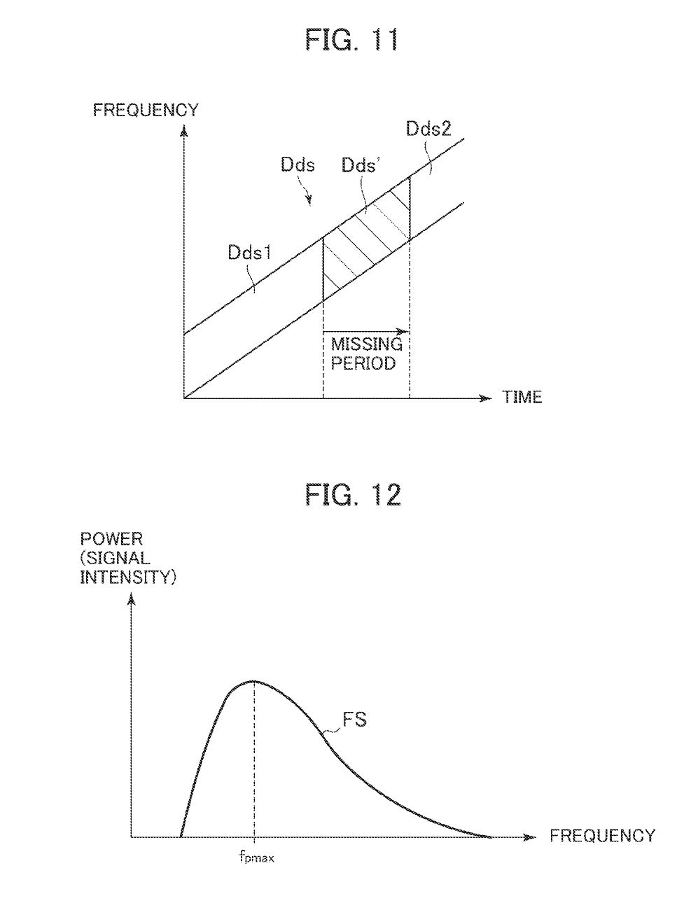

As illustrated in FIG. 10, the extrapolation process supplements estimation data Dds' for missing part of Doppler spectrum data Dds. FIG. 11 supposes that Dds1 denotes Doppler spectrum data Dds before the beginning of the missing period and Dds2 denotes Doppler spectrum data Dds after the end of the missing period. Then, the estimation data Dds' is continuous with the Doppler spectrum data Dds1 and the Doppler spectrum data Dds2. Accordingly, high-quality data can be ensured even if Doppler spectrum data Dds varies with the time as illustrated in FIGS. 9 through 11. The extrapolation process can supplement data immediately after the beginning of a missing period and, unlike an interpolation process, need not wait until the missing period ends, for example. The extrapolation process can supplement the missing part without delay.

After being supplemented with the missing part by the signal estimation unit 425, Doppler spectrum data Dds is output to the display control unit 5 and the IFFT processing unit 426.

The display control unit 5 allows the display unit 6 to display a Doppler image generated based on the Doppler spectrum data that is directly supplied from the signal estimation unit 425 or the FFT processing unit 424.

The IFFT processing unit 426 performs an IFFT process on the Doppler spectrum data supplied from the signal estimation unit 425 or the FFT processing unit 424. The IFFT-processed data is output to the audio processing unit 427.

The audio processing unit 427 performs an audio process on the data supplied from the IFFT processing unit 426 and outputs a signal to the speaker. The speaker 9 outputs Doppler sound. As described above, the signal estimation unit 425 performs the extrapolation process to supplement a missing part without delay even if the Doppler sound is output based on the Doppler spectrum data output from the signal estimation unit 425. Therefore, the Doppler sound can be output without delay.

If the signal estimation unit 425 performs no process, the wall filter unit 422 may supply data to the audio processing unit 427 and output the Doppler sound.

The following describes modifications of the embodiment. A first modification will be described. The signal estimation unit 425 may perform an extrapolation process based on a temporal change in the frequency for the Doppler spectrum data. The signal estimation unit 425 is not limited to performing an extrapolation process based on a temporal change in the average frequency for the Doppler spectrum data as described above. As illustrated in FIG. 12, for example, the signal estimation unit 425 may perform an extrapolation process based on a temporal change in frequency fpmax having the peak power in frequency spectrum FS for the Doppler spectrum data. Also in this case, for example, the signal estimation unit 425 uses linear function F as an extrapolation function so that linear function F is found from two points in a data string on a temporal change line (not shown) for the frequency fpmax.

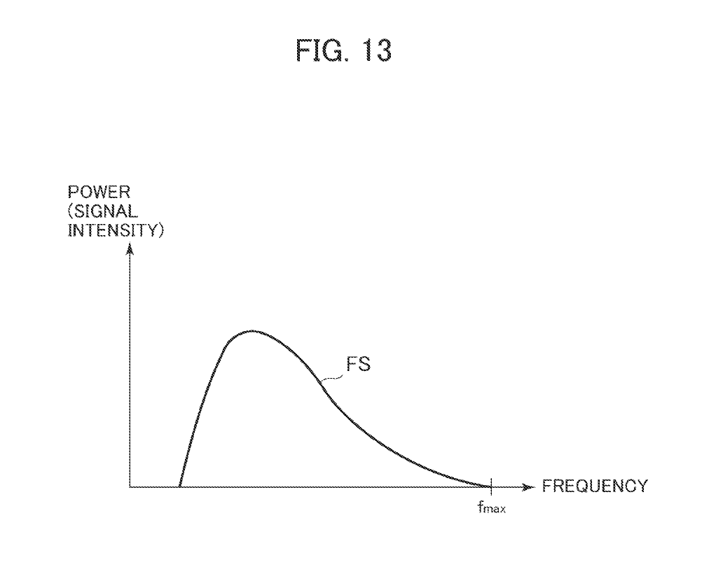

A second modification will be described. As illustrated in FIG. 13, the signal estimation unit 425 may perform an extrapolation process based on a temporal change of maximum frequency fmax in frequency spectrum FS for the Doppler spectrum data. Also in this case, for example, the signal estimation unit 425 uses a linear function as an extrapolation function so that the linear function is found from two points in a data string on a temporal change line (not shown) for the maximum frequency fmax.

While there have been described specific preferred embodiments of the invention, it is to be distinctly understood that the invention is not limited thereto but may be otherwise variously embodied within the spirit and scope of the invention.

Many widely different embodiments of the invention may be configured without departing from the spirit and the scope of the present invention. It should be understood that the present invention is not limited to the specific embodiments described in the specification, except as defined in the appended claims.

### INDUSTRIAL APPLICABILITY

The present invention is applied to the ultrasound diagnostic apparatus which estimates missing part of the Doppler spectrum signal, and the apparatus can produce high-quality Doppler spectrum signals.

1. An ultrasound diagnostic apparatus comprising:
an ultrasound probe that performs ultrasound transmission/reception in Doppler mode and ultrasound transmission/reception in other mode than Doppler mode; and
a Doppler processing unit that performs quadrature detection on an echo signal generated from the ultrasound transmission/reception for Doppler mode and then generates a Doppler spectrum signal,
wherein the Doppler processing unit includes a signal estimation unit that performs an extrapolation process to estimate missing part of the Doppler spectrum signal resulting from the ultrasound transmission/reception for other mode.

2. The ultrasound diagnostic apparatus according to claim 1,
wherein the signal estimation unit performs an extrapolation process based on a temporal change of a frequency for the Doppler spectrum signal.

3. The ultrasound diagnostic apparatus according to claim 2,
wherein a temporal change of a frequency for the Doppler spectrum signal is equivalent to a temporal change of an average frequency for a frequency spectrum for the Doppler spectrum signal.

4. The ultrasound diagnostic apparatus according to claim 2,
wherein a temporal change of a frequency for the Doppler spectrum signal is equivalent to a temporal change of a frequency having peak power in a frequency spectrum for the Doppler spectrum signal.

5. The ultrasound diagnostic apparatus according to claim 2,
wherein a temporal change of a frequency for the Doppler spectrum signal is equivalent to a temporal change of a maximum frequency in a frequency spectrum for the Doppler spectrum signal.

6. The ultrasound diagnostic apparatus according to claim 1,
wherein the signal estimation unit settles a data interval according to a subject region, the data interval being configured to find an extrapolation function used for an extrapolation process.

7. The ultrasound diagnostic apparatus according to claim 2,
wherein the signal estimation unit settles a data interval according to a subject region, the data interval being configured to find an extrapolation function used for an extrapolation process.

8. The ultrasound diagnostic apparatus according to claim 3,
wherein the signal estimation unit settles a data interval according to a subject region, the data interval being configured to find an extrapolation function used for an extrapolation process.

9. The ultrasound diagnostic apparatus according to claim 4,
wherein the signal estimation unit settles a data interval according to a subject region, the data interval being configured to find an extrapolation function used for an extrapolation process.

10. The ultrasound diagnostic apparatus according to claim 5,
wherein the signal estimation unit settles a data interval according to a subject region, the data interval being configured to find an extrapolation function used for an extrapolation process.

11. The ultrasound diagnostic apparatus according to claim 1,
wherein the Doppler processing unit uses a Fourier transform process to generate the Doppler spectrum signal.

12. The ultrasound diagnostic apparatus according to claim 2,
wherein the Doppler processing unit uses a Fourier transform process to generate the Doppler spectrum signal.

13. The ultrasound diagnostic apparatus according to claim 3,
wherein the Doppler processing unit uses a Fourier transform process to generate the Doppler spectrum signal.

14. The ultrasound diagnostic apparatus according to claim 4,
wherein the Doppler processing unit uses a Fourier transform process to generate the Doppler spectrum signal.

15. The ultrasound diagnostic apparatus according to claim 5,
wherein the Doppler processing unit uses a Fourier transform process to generate the Doppler spectrum signal.

16. The ultrasound diagnostic apparatus according to claim 6,
wherein the Doppler processing unit uses a Fourier transform process to generate the Doppler spectrum signal.

17. A method for generating a Doppler spectrum signal comprising the steps of:
performing ultrasound transmission/reception in Doppler mode and ultrasound transmission/reception in other mode than Doppler mode;
performing quadrature detection on an echo signal generated from the ultrasound transmission/reception for Doppler mode;
generating a Doppler spectrum signal; and
performing an extrapolation process to estimate missing part of the Doppler spectrum signal resulting from the ultrasound transmission/reception for other mode.
